(19) United States
(12) Patent Application Publication
(10) Pub. No.: US 2002/0045277 A1
(43) Pub. Date: Apr. 18, 2002

(54) Process for detecting biological molecules

(76) Inventors: Beate Schmid, Bonn (DE); Daniel Hoffmann, Bonn (DE); Stefan Wefing, Bonn (DE); Volker Schnaible, Bonn (DE); Eckhard Quandt, Bonn (DE); Michael Tewes, Hurth (DE); Michael Famulok, Bonn (DE)

Correspondence Address: LOWE HAUPTMAN GOPSTEIN, GILMAN AND BERNER LLP, SUITE 310, 1700 DIAGONAL ROAD, ALEXANDRIA, VA, 22314

(21) Appl. No.: 09/973,993

(22) Filed: Oct. 11, 2001

(30) Foreign Application Priority Data
Oct. 12, 2000 (DE) 100 50 632.1

(51) Int. Cl. C12Q 1/68; G01N 33/543

(52) U.S. Cl. 436/518; 435/6

(57) ABSTRACT

Process for qualitative and quantitative detection of an analyte present in a biological sample and in molecular form, in which process a sensor is provided with a substrate binding the analyte and in which in a first process step the sensor is loaded with the analyte and, in a subsequent process step, the total analyte mass bound by the sensor is measured, before in a further step the molecular weight of the analyte forming the total mass is measured.

The present invention relates to a process for qualitative and quantitative detection of an analyte present in a biological sample and in molecular form, in which process a sensor is provided with a substrate binding the analyte.

Following the decoding of the human genome and other genomes, techniques have been developed in order to measure rapidly and simply mRNA expression of cells under various conditions, for example diseased and healthy states. However, mRNA expression does not give a complete picture of cellular events at the crucial protein level: protein expression is regulated not only via expression of mRNA but also via other mechanisms and, in addition, proteins frequently display post-translational modifications (PTMs) which are specific for a tissue type or a cellular state and which cannot be studied at the mRNA level. The proteome of a cell or a tissue, that is to say the types and amounts of all proteins present therein, therefore provides more information than the mRNA expression pattern and allows a more accurate description of the state of the cell or the tissue. Therefore, proteome analyses are very useful in medical diagnostics, in finding target proteins for therapeutic active substances and in the development of the active substances themselves.

The only method for proteome analysis in use at present is a two-step process. Here, the proteome is first fractionated according to isoelectric point and size of the proteins present with the aid of two-dimensional gel electrophoresis (2DGE). The proteins are then characterized by mass spectrometry (MS). This method has several disadvantages. The greatest disadvantages are that the reproducible preparation of high-solution gels is complicated, that certain proteins (for example very basic or very large proteins) are cut out most of the time, that weakly expressed proteins are not detected, that assigning spots unambiguously to individual proteins is not always possible and that fractionation on the gel usually leads to unfolding of the proteins, making subsequent studies on function and 3D structure more difficult.

It is the object of the invention to provide a practical process which can be used to analyse biological molecules such as proteomes, for example, simply and conveniently.

This object is achieved by using a process according to the preamble to Claim 1, which process is characterized in that in a first process step the sensor is loaded with the analyte, that in a subsequent process step the total analyte mass bound by the sensor is measured, before in a further step the molecular weight of the analyte forming the total mass is measured.

The invention thus relates to a process for rapidly detecting and identifying randomly selected molecules in biological samples and determining the amounts of those molecules in these samples. In this connection, it is possible to detect individual types of molecules but also whole classes of molecules, for example proteins sharing a particular domain. The process may also be used for elucidating interactions between proteins or between proteins and other molecules. The method is suitable for rapid and reliable proteome analysis and is of high economic value in particular for medical practice and the pharmaceutical industry.

The process of the invention allows specific and rapid analysis of specific proteome sections chosen by the user, avoiding the disadvantages of the traditional process. Since the process of the invention works under native conditions, proteins remain structurally and functionally intact. In addition, the detection has no upper mass limit. The process of the invention therefore allows studying of protein complexes and the like and thus makes it possible to characterize protein interactions and other interaction networks. The process of the invention thus opens novel applications.

The process of the invention comprises the following steps or a part sequence of these steps:

1. Selection of ligands:

Starting from a biomedical question, suitable proteins, protein domains, epitopes, metabolites or the like (“ligands”), whose proportion of the proteome or cell contents is to be determined, are selected. Selection criteria may be, for example, the key position within metabolic or regulatory webs or the strong mRNA expression. The selection is carried out manually or by bioinformatic analysis of biological networks or other data.

2. Preparation of ligands:

The selected ligands are prepared according to processes known per se. Thus, proteins are expressed and purified. It other ligands (e.g. metabolites) have been selected in step 1, these molecules are provided, for example, by chemical synthesis or extraction from biological material.

3. Preparation of receptors:

“Receptors” are prepared, which bind tightly to the selected ligands. Examples of possible receptors are aptamers of oligonucleotides or antibodies. The preparation of such receptors is known per se (see, for example, Tuerk & Gold 1990 Science 249:505, Ellington & Szostak 1990 Nature 346:818).

4. Preparation of sensor arrangement:

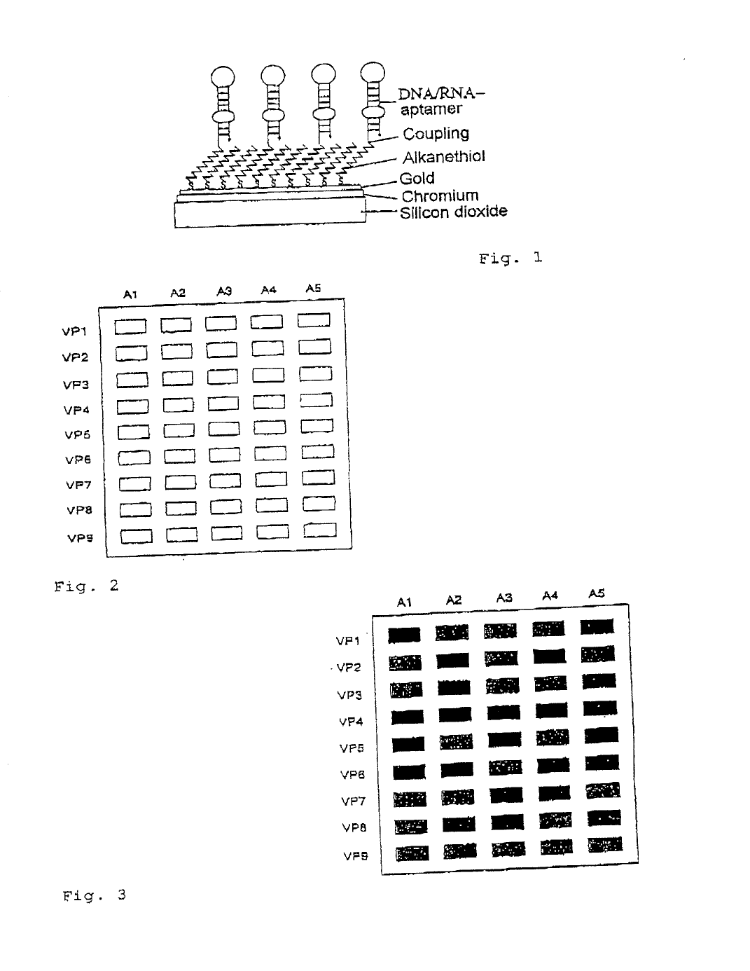

The sensors used are microbalances, for example “Love wave” sensors (Harding et al 1997 Sensors and Actuators A 61:279; FIG. 1). It is possible to arrange a plurality of sensors as an array on a shared base. In the case of Love wave sensors, the mass of an applied analyte is measured via changes in the frequency spectrum of surface shear waves of the sensor, for example as a shift in resonance frequencies or phases. This change results from ligands attaching to the receptors which are immobilized on the sensor (step 5). As an alternative to Love wave sensors, sensors detecting changes in other physical parameters (fluorescence, electrochemical changes) may also be employed.

5. Immobilization of receptors:

The receptors are coupled to self-assembled monolayers (SAMs). Typically, SAMs are prepared from substituted alkanethiols for a gold-coated sensor (step 4) or from silane compounds for a sensor having a silicon dioxide surface. The SAM molecules bind covalently to the surface of the sensor from step 4. The aptamers for their part are coupled terminally to the SAM molecules, thereby forming, for example, carboxylic acid-amide-, carboxylic acid-ester- or phosphoric acid-amide-type bonds. These couplings are reversible, and this facilitates refuse of the sensor and can be utilized for the measurement process (step 6).

6. Measurement of total ligand mass:

The analyte, for example a cell lysate, is pipetted onto the sensors. Washing removes those analyte components which bind unspecifically to the receptors. The masses of the ligands bound to the sensor via receptors are measured via the change in the frequency spectrum of the surface waves of the Love wave sensor or via the change in other suitable measurement parameters when using a different sensor. When measuring the total mass, attention must be paid especially to two sources of error:

First, parts of the sample sometimes stick unspecifically to the SAM and thus falsify the measurement; secondly, for very small and light ligands, the measured signal may disappear in the noise. In the first case, sensitivity may be increased by applying the following process: in step 5, the receptors are coupled to the base via cleavable bonds. After washing a first time and measuring, the receptor/ligand complexes are removed by cleaving the said bond and the sensor surface is washed a second time. The supernatant then contains the receptor/ligand complexes, while the analyte parts which bind unspecifically remain on the sensor. The mass of the receptor/ligand complexes is given by the difference in masses before and after the second washing.

If the low ligand mass is the main source of error, the sensitivity may be increased as follows: a molecular signal enhancement is achieved by using specific receptors which are catalytically active and whose activity is regulated allosterically by the ligand. The catalytic activity may, for example, consist of the receptor, after ligand binding, cleaving off a part of itself. If the ligand is light but the part cleaved off is heavy, then the signal is enhanced: after ligand binding, the mass markedly decreases.

7. Measurement of molecular masses and determination of the amounts of ligands:

The more specific the receptors, the more accurately defined is the ensemble of ligands attached thereto. Thus, highly specific receptors are used for detecting ligands which differ only slightly, for example by a post-translational modification. In contrast, receptors with lower specificity are used for detecting novel ligands. Thus, for example, receptors recognizing only a small epitope on protein ligands are used for detecting proteins having this epitope. In both cases, the ligands actually bound are identified in a second analytical step with the aid of mass spectrometry. For this purpose, the molecular masses of ligands or the masses of ligand fragments are measured. Fragmentation is achieved using various chemical and physical techniques. In the case of proteins, it is achieved by specific chemical breakdown, for example by specific enzymic proteolysis and subsequent decomposition of the peptides in a mass spectrometer (Gatlin et al 2000 Anal Chem 72:757; Chaurand et al 1999 J Am Soc Mass Spec 10:91). In order to avoid sample losses during transfer to the mass spectrometer, proteolysis and sample reading by the mass spectrometer take place directly on the sensor which was used in step 6 to measure the total mass. In the simplest case of a sensor being loaded with exactly one ligand species, the amount of bound ligands is calculated as follows: step 6 determines the total mass M of all ligand molecules bound to the sensor and step 7 determines the mass m of the individual ligand molecule. Thus, the amount of bound ligand molecules M=m. If two ligand types are present, it is possible to use the Love wave sensor for determining the total mass M of all ligand molecules of both types and to use mass spectrometry (see, for example, Cohen et al 2000 Anal Chem 72:574) for determining both the masses m1 and m2 of the individual ligand molecules and the ratio R of the amounts of ligands of the second and first type; this gives the amount of the first type M=(m1+R_m2). For three or more types, the procedure is analogous. The process of the invention thus allows direct quantification of the amounts of any types of ligands.

8. Comparison with reference:

The concentrations of the molecules bound and types present are compared with corresponding data from reference samples. For example, data from a lysate of diseased cells are compared with those from healthy tissue. Comparison of the measured values from step 6 with corresponding values of reference samples of known ligand concentration allows determination of the ligand concentration in the biological sample. For some applications, such as the finding of target proteins for active substances for example, steps 1 to 8 are generally repeated several times with new ligands and receptors. In order to achieve high process efficiency, it is advantageous to minimize the number of repeats, in particular running the comparatively complicated step 2. For this purpose, the present knowledge about metabolic and regulatory networks and other experimental data which provide information on differences in the frequency of proteins or other molecules between healthy and diseased tissue is used. Starting from these data, it is possible, for example by repeating steps 1 to 8 several times, to find those ligands whose concentrations in healthy and diseased tissue display the greatest difference. These ligands are suitable targets for therapies or interact directly with such target molecules. In an ideal case, the receptors to which the said ligands have bound are suitable as active substances.

On the basis of the figures, an exemplary embodiment of the invention is illustrated below. In the figures,

FIG. 1 shows a cross section through a Love wave sensor with aptamers as immobilized receptors;

FIG. 2 shows a scheme of a microbalance array in the application example. Here, five aptamers each against each of the nine viral proteins VP1, . . . VP9 are produced. Each array thus has 45 aptamer sensors;

FIG. 3 shows a mass measurement using the microbalance: the darker the shade of grey, the higher the total mass on the sensor. The greatest increase in mass is measured on those sensors on which aptamer against VP4 was immobilized. There, VP4 binds completed with TF. Thus, VP4 is the desired viral protein. In contrast, aptamers A3 and A4 bind VP4 competitively to TF. These aptamers therefore are candidates for antiviral active substances.

The following example shows how an embodiment of the above-described process makes a novel application possible. In this example, the aim is to develop an active substance against a virus pathogenic in humans. One (VPX) of the viral proteins (VPs) interacts with a human transcription factor (TF) and thereby enhances production of virus particles. Although all proteins of the virus are known, it is not known which of the VPs interacts with which TF. In the search for an active substance, the first part of the problem is therefore identification of VPX and the TF interacting therewith. Using the process of the invention, this problem is solved, for example, as follows:

In the first step, aptamers (receptors) against all VPs (ligands) are produced, in fact a plurality of aptamers against each VP are produced.

In the next step, the aptamers are immobilized on an arrangement of sensors, that is, with one aptamer type each per sensor (FIG. 2). The sensors are layered Love wave sensors (FIG. 1): on an SiO₂ substrate a chromium layer and, on top of this, in turn a gold layer are applied; a self-assembled monolayer composed of substituted alkanethiols is covalently bound to the gold layer; finally, DNA aptamers are coupled to the alkanethiols via a carboxylic acid-amide bond.

In the next step, the lysate of virus-infected cells is pipetted on each sensor of the arrangement and molecules binding weakly and unspecifically are removed by washing. For example, when increasing the concentration of a detergent or when increasing the salt concentration, weakly binding molecules are removed first. In this connection, the Love wave sensor signal serves to monitor the washing success: after the weakly bound molecules have been removed from the sensor surface, the measured signal changes only slowly with increasing detergent or salt concentration.

In the next step, aptamers on one sensor or on several sensors of the arrangement bind VPs, partly completed with TFs, and this can be measured via the frequency change of shear waves on the surface of the said Love wave sensors. The measured values are normalized by adding in each case VPs of known concentration, so that all aptamers are saturated with VPs. Subsequently, the total ligand mass on each sensor is measured again. The mass difference compared with the previous measurement is proportional to the number of receptors which have not been occupied by ligands from the lysate. The smaller this number, the higher the ligand concentration in the lysate.

After saturating the aptamers with VPs in step 4, the total mass on some sensors is greater than the mass of VPs alone. Among the ligands on these sensors is probably the desired VPX, completed with TFs (FIG. 3). The actual identity of the ligands is determined by mass spectrometry. For this purpose, the ligands on the said sensors are digested by specific proteolysis, for example with trypsin. The masses of the peptide fragments are then measured in the mass spectrometer and the proteins, in particular TFs, are identified by database search (peptide mass fingerprint). Thus, VPX and the TFs interacting therewith are known and the first part of the problem has been solved.

In step 1, a plurality of aptamers against VPX, which differ, for example, in their binding site on VPX, were produced. Aptamers which compete with TF for the same binding site on VPX may ideally be used as active substances against the virus. In steps 4 and 5, such aptamers are distinguished by the fact that the sensors on which the said aptamers have been immobilized bind VPX but no complexes of VPX and TFs (FIG. 3).

1. Process for qualitative and quantitative detection of an analyte present in a biological sample and in molecular form, in which process a sensor is provided with a substrate binding the analyte, characterized in that in a first process step the sensor is loaded with the analyte, that in a subsequent process step the total analyte mass bound by the sensor is measured, before in a further step the molecular weight of the analyte forming the total mass is measured.

2. Process according to claim 1, characterized in that the total mass is measured by means of a microbalance, in particular by means of a Love wave sensor.

3. Process according to either of claims 1 and 2, characterized in that a plurality of sensors is arranged in a joint sensor array.

4. Process according to any of the preceding claims, characterized in that the molecular weight of the analyte is measured by means of a mass spectrometer.

5. Process according to claim 4, characterized in that the total mass bound in the sensor is analysed using a mass spectrometer.

6. Process according to any of the preceding claims, characterized in that a plurality of analytes is measured synchronously.

7. Process according to any of the preceding claims, characterized in that various substrates which bind the same analyte in various ways are used in the sensor array.

8. Process according to any of the preceding claims, characterized in that various substrates which bind various analytes are used in the sensor array.

9. Process according to any of the preceding claims, characterized in that the analyte is part of a molecular complex composed of a plurality of molecules.

10. Process according to any of the preceding claims, characterized in that the substrate is reversibly immobilized on the sensor.

11. Process according to any of the preceding claims, characterized in that the measurement is carried out under physiological conditions.

12. Process according to any of the preceding claims, characterized in that the substrate is selected such that it binds the analyte on an epitope.

13. Process according to any of the preceding claims, characterized in that the quantitative ratios of different types of analyte molecules are determined via the signal intensities in the mass spectrum.

14. Process according to any of the preceding claims, characterized in that the absolute amounts of different analyte molecules are determined by the total analyte mass on the sensor, the molecular masses and the quantitative ratios.

15. Process according to any of the preceding claims, characterized in that the proportion of substrate loaded with analyte molecules is determined by differential measurement between the actual analyte and additional saturation of the substrate with a calibration sample of known analyte concentration.

16. Process according to any of the preceding claims, characterized in that a substrate is chosen from oligonucleotides or proteins.

17. Process according to any of the preceding claims, characterized in that the total mass signal measured during washing of the sensor is used for optimizing sample preparation for mass spectrometry.

18. Process according to any of the preceding claims, characterized in that comparative measurements are carried out on biological samples of different origin or under various conditions.

19. Process according to any of the preceding claims, characterized in that measured signals together with known biomolecular interactions and with comparative measurements are used in order to select novel substrates which bind to other analytes and then new measurements using these substrates are carried out.

20. Device for carrying out the process according to any of the preceding claims.
